(19) United States
(12) Patent Application Publication
(10) Pub. No.: US 2009/0042204 A1
(43) Pub. Date: Feb. 12, 2009

(54) ACNE LESION BIOMARKERS AND MODULATORS THEREOF

(76) Inventor: DIANE THIBOUTOT, Hershey, PA (US)

Correspondence Address: BUCHANAN, INGERSOLL & ROONEY PC, POST OFFICE BOX 1404, ALEXANDRIA, VA, 22313-1404, US

(21) Appl. No.: 12/168,633

(22) Filed: Jul. 7, 2008

Related U.S. Application Data
(63) Continuation of application No. PCT/EP2007/050122, filed on Jan. 5, 2007.
(60) Provisional application No. 60/756,212, filed on Jan. 5, 2006.

(51) Int. Cl. C12Q 1/68; G01N 33/50

(52) U.S. Cl. 435/6; 436/94

(57) ABSTRACT

The present invention relates to acne lesions biomarkers/genes expression products pattern and particularly inflammatory acne lesions biomarkers and their uses, modulators thereof and the use of modulators for acne treatment or associated disorders. Invention also concerns in vitro diagnostic methods.

### CROSS-REFERENCE TO PROVISIONAL/PCT APPLICATIONS

This application claims priority under 35 U.S.C. § 119 of U.S. Provisional Application No. 60/756,212, filed Jan. 5, 2006, and is a National Phase/Continuation of PCT/EP 2006/050122, filed Jan. 5, 2007, and designating the United States Jul. 12, 2007, as WO 2007/077257 A2, each hereby expressly incorporated by reference in its entirety and each assigned to the assignee hereof.

### BACKGROUND OF THE INVENTION

1. Technical Field of the Invention

The present invention relates to identification of acne lesion biomarkers/genes expression products pattern and particularly inflammatory acne lesion biomarkers and applications of same in screening methods, modulators thereof and the use of modulators for acne treatment or acne associated disorders. This invention also relates to in vitro diagnoses of these diseases.

2. Description of Background and/or Related and/or Prior Art

Acne is the most common skin condition affecting millions of people worldwide. Patients with severe acne frequently face significant psychological and emotional problems due to the scarring associated with the disease. The pathogenesis of acne vulgaris is complex and incompletely understood.

The pathogenesis of acne has been linked to multiple factors such as increased sebum production, inflammation, follicular hyperkeratinization and the action of Propionibacterium acnes within the follicle.

Inflammation is a key component of the pathogenesis of acne. An immunological reaction to the gram-positive microbe P. acnes may play a major role in the initiation of the inflammatory reaction (De Young et al, 1984; Jappe et al, 2002). Recently published studies also implicate Toll Like receptor 2 (TLR-2) in inflammatory acne.

Propionibacterium acnes triggers pro-inflammatory cytokine release from inflammatory cells via activation of TLR-2, which in turn initiates an intracellular signaling cascade resulting in the transcription of genes such as interleukin-12 and interleukin-8 (Kim et al, 2002). Furthermore, viable P. acnes and not heat-killed organisms, can stimulate the release of cytokines such as IL-1β, granulocyte/macrophage colony stimulating fraction (GM-CSF) and IL-8 (Nagy et al, 2005; Schaller et al, 2005).

While the exact initiating event causing acne still remains a mystery, there exists a debate as to whether hyperkeratinization of the follicular duct precedes the influx of inflammatory cells or vice versa. Recent studies support the later hypothesis by demonstrating that an increase in IL-1 activity occurs prior to the hyperproliferation around uninvolved follicles and this triggers the ‘keratinocyte activation cycle’. (Freedberg et al, 2001; Jeremy et al, 2003)

Recent reports demonstrate that the skin expresses various anti-microbial peptides in response to the proliferation of pathogens as part of cutaneous innate immunity (Braff et al, 2005; Schroder, 2004; Selsted and Ouellette, 2005). Important amongst this group of anti-microbial agents include members of the human β defensin family and granulysin-derived peptides (Deng et al, 2005; Harder et al, 2004; McInturff et al, 2005). Human β defensin-1 and 2 (HBD-1 and HBD-2) are expressed in the pilosebaceous unit and their expression is upregulated in acne lesions (Chronnell et al, 2001). Recent studies have also discovered that select strains of P. acnes can activate HBD-2 through TLRs further confirming the importance of these peptides in inflammatory acne (Nagy, et al., 2005).

Acne research during the last 25 years has significantly increased understanding about the etiological factors giving rise to acne. With the advent of gene array expression profiling however, new opportunities have arisen to re-examine the disease and potentially identify novel targets in its treatment.

Need therefore exists to identify the specific genes expressed in inflammatory acne lesions compared to normal skin from acne patients and to test the hypothesis that differences in gene expression exist from normal skin from acne patients and skin from subjects without acne that may account for the predisposition to the disease and when acne is revealed to establish novel therapeutics able to modulate/regulate genes expression.

### SUMMARY OF THE INVENTION

To understand the specific genes involved in inflammatory acne, gene expression profiling has now been carried out in acne patients. Skin biopsies were obtained from an inflammatory papule and from normal skin in 6 patients with acne. Biopsies were also taken from normal skin of 6 subjects without acne. Gene array expression profiling was conducted using Affymetrix U133A chips comparing lesional to non-lesional skin in acne patients and comparing non-lesional skin from acne patients to skin from normal subjects. Within the acne patients 211 genes are upregulated in lesional skin compared to non-lesional skin. A significant proportion of these genes are involved in pathways that regulate inflammation and extra-cellular matrix remodeling and they include matrix metalloproteinase's 1 & 3, interleukin-8, human β defensin 4 and granzyme B. These data indicate a prominent role of matrix metalloproteinases, inflammatory cytokines and anti-microbial peptides in acne lesions. The studies of the present invention are the first describing the comprehensive changes in gene expression in inflammatory acne lesions and as such are considered to be of value in identifying potential therapeutic targets in inflammatory acne.

The present invention features targeting the genes as mentioned above and/or expression products of the genes to prevent and/or treat acne as well as acne associated disorders (e.g., hyperseborrhoea).

By “acne” are intended all acne forms, especially simple acne, comedonic acne, papulopustular acne, papulocomedonic acne, nodulocystic acne, acne conglobata, cheloid acne of the nape of the neck, recurrent miliary acne, necrotic acne, neonatal acne, occupational acne, acne rosacea, senile acne, solar acne and medication-related acne.

In the context of the present invention “gene” refers to nucleic acid or nucleotide sequence encoding for a protein/biomarker expression. Said gene is also considered as a gene “target” for which a modulator is sought.

When the target is preferably a human gene or its expression product, the invention might use cells expressing the gene or the associated protein/biomarker by genomic incorporation or by transitory gene expression encoding protein.

Corresponding human sequence references are indicated in Tables 1 and 2.

### BRIEF DESCRIPTION OF THE DRAWINGS

Table 1 is a list of genes regulated in acne lesions when compared to normal skin.

Table 2 is a list of genes product regulated in acne lesions compared to normal skin.

Table 3 is a table identifying enhanced expression of genes involved in inflammation and matrix remodeling in acne lesions.

Table 4 is a list of genes and biomarkers/gene expression product for modular screening.

FIG. 1 is a graph illustrating the hierarchical clustering of genes involved in inflammation.

FIG. 2 shows confirming changes in gene expression pattern by immunohistochemistry.

FIG. 3 shows the immunochemistry staining of MMP-1 in clinically normal skin and inflammatory acne.

### DETAILED DESCRIPTION OF BEST MODE AND SPECIFIC/PREFERRED EMBODIMENTS OF THE INVENTION

The present invention features acne lesion biomarkers and/or gene expression products as acne lesion biomarkers selected from the following list:

Matrix metalloproteinase 1 (MMP1); matrix metalloproteinase 3 (MMP3); interleukin 8 (IL8); beta 4 defensin (DEFB4); skin-derived (SKALP) protease inhibitor 3 (PI3); chemokine (C-X-C motif) ligand 2 (CXCL2); apolipoprotein B mRNA editing enzyme (APOBEC3A); superoxide dismutase 2, mitochondrial (SOD2); granzyme B (GZMB); S100 calcium binding protein A9 (calgranulin B) (S100A9); chemokine (C-C motif) receptor 1 (CCR1); heparanase (HPSE); serum amyloid A2 (SAA2); leukotriene B4 receptor (LTB4R); tumor necrosis factor receptor superfamily member 1B (TNFRSF1B); complement component 3a receptor 1 (C3AR1); G protein-coupled receptor 65 (GPR65); baculoviral IAP repeat-containing 1 (BIRC1); CD14 antigen (CD14); serum/glucocorticoid regulated kinase (SGK); CD28 antigen (Tp44) (CD28); apoptosis caspase activation inhibitor (AVEN); TNF receptor-associated factor 3 (TRAF3); aldo-keto reductase family 1, member B10 (AKR1B10); phospholipase A2, group IIA (platelets, synovial fluid) (PLA2G2A); UDP glycosyltransferase 1 family, polypeptide A10 (UGT A10); phospholipid scramblase 1 (PLSCR1); serum amyloid A2 (SAA2); fatty acid binding protein 5 (psoriasis-associated) (FABP5); arachidonate 5-lipoxygenase (ALOX5); phosphoinositide-3-kinase, catalytic, delta polypeptide (PIK3CD); secretoglobin, family 1D, member 2 (SCGB1D2); secretoglobin, family 2A, member 1 (SCGB2A1); transmembrane 4 superfamily member 3 (TM4SF3); mutS homolog 5 (E. coli) (MSH5); secretoglobin, family 2A, member 2 (SCGB2A2); mutS homolog 5 (E. coli) (MSH5); frizzled-related protein (FRZB); hypothetical protein MGC11242 (MGC11242); SRY (sex determining region Y)-box 10 (SOX10); keratin 18 (KRT18); lipase hepatic (LIPC); coagulation factor X (F10); hypothetical protein FLJ20280 (FLJ20280); fast troponin C2, (TNNC2); KDEL endoplasmic reticulum protein retention receptor 3 (KDELR3); KIAA0514; DKFZP5640243 protein; nuclear receptor subfamily 1, group D, member 1 (NR1D1); CD47 antigen (Rh-related antigen, integrin-associated signal transducer) (CD47); Thy1 cell surface antigen (Thy1); selectin P ligand (SELPG); TIMP metallopeptidase inhibitor 1 (TIMP1); chemokine (C-C motif) ligand 21 (CCL21); S100 calcium binding protein A9 (calgranulin B) (S100A9); chemokine (C-C motif) receptor 2 ///chemokine (C-C motif) receptor 2 (CCR2); chemokine (C-C motif) receptor 5 (CCR5); selectin L (lymphocyte adhesion molecule 1)(SELL).

Particularly, these biomarkers are inflammatory acne lesion biomarkers.

In the context of the present invention, it is understood as biomarker a characteristic that is objectively measured and evaluated as an indicator of normal biologic processes, pathogenic processes, or pharmacologic responses to a therapeutic intervention (NIH definition).

Therefore, biomarkers are employed to indicate or measure a biological process (for instance, levels of a specific protein in blood or fluids, genetic mutations, or abnormalities observed in tests). Detecting biomarkers specific to a disease can aid in the identification, diagnosis, and treatment of affected individuals and individuals who may be at risk but do not yet exhibit symptoms.

In addition, the present invention relates to the said gene expression product as “biological targets”. By target is understood an enzyme, a receptor, other protein or mRNA that can be modified by an external stimulus. The definition is context-dependent and can refer to the biological target of a pharmacologically active drug compound, or the receptor target of a hormone. The implication is that a molecule is “hit” by a signal/stimulus and its behavior is thereby changed.

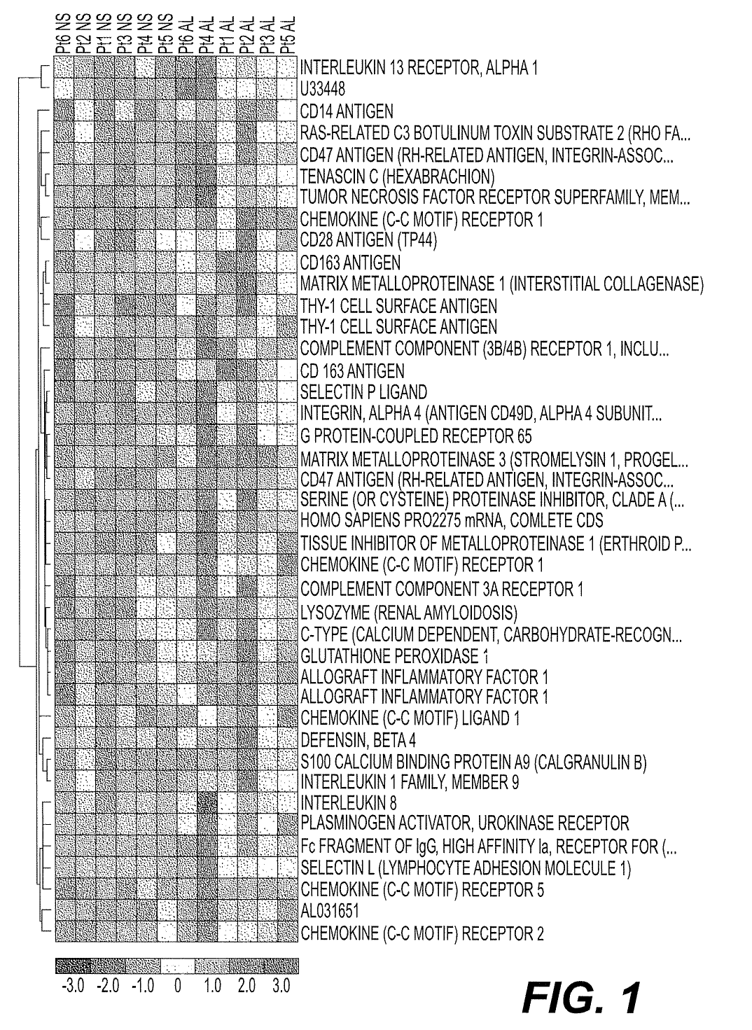

According to the present invention, targets of interest are those disclosed in Tables 1 and 2, and FIG. 1 and more specifically above mentioned expression products.

### Diagnostic Methods:

One embodiment of the present invention encompasses an acne diagnostic method or an acne disease or acne associated disorders evolution follow-up method by using the said biomarkers/gene expression products here disclosed. Therefore, said method comprises the step of comparing expression of genes or biomarkers/genes expression products activity indicated in Tables 1 and 2 and FIG. 1 in a patient biological sample with subject “control” sample.

Gene expression products/Biomarkers (e.g., Proteins) might be determined by any appropriate methods such as western-blot, IHC, MAS spectrometry analysis (MAldi-TOF and LC/MS analysis), Radioimmunoassay (RIA), Elisa or by any other methods well known by one skilled in the art or by mRNA dosage by any appropriate methods well known to one skilled in the art.

It is understood by subject “control” is a subject in healthy conditions or in non-involved skin of acne conditions.

In the context of an acne disease or acne associated disorders evolution follow-up method, the “control” subject is understood as the same subject where biological sample was taken at a different time and preferentially at the beginning of the treatment or before the treatment (Baseline). The comparison of gene expression or Biomarkers/gene expression products levels, is a tool to determine the product efficacy and decide whether or not continue the treatment with the same product.

Another embodiment of this invention is an in vitro determination method of patient sensitivity to develop acne lesions and/or acne associated disorders, which comprises the step of comparing above mentioned genes expression levels or genes expression products levels or activity of biomarkers/gene expression products in a patient biological sample with a subject “control” biological sample.

Again, biomarkers and/or gene expression products levels might be measured by any appropriate methods such as western-blot, IHC, MAS spectrometry analysis (MAldi-TOF and LC/MS analysis), Radioimmunoassay (RIA), Elisa or by any other methods well known to one skilled in the art and for example by ELISA dosage or by mRNA dosage by any appropriate methods well known to one skilled in the art.

In this case, patient is asymptomatic and presents any acne visible symptoms. Therefore the “control” subject is one from healthy reference population.

In the context of above mentioned diagnostic methods, the biological sample might be any biological fluid sample (sebum, blood, urine, plasma . . . ) or any sample extract by biopsy and is preferentially a skin sample.

### Screening Methods:

Another embodiment of the present invention is an in vitro screening method of drug candidates (or family lead compound) susceptible of preventing and/or treating acne as well as acne associated disorders (e.g., hyperseborrhoea). The said method comprises the step of determining the drug candidate capacity to modulate (e.g., down regulated or up regulate) said genes expression and/or said biomarkers/gene expression products levels mentioned in Table 3 and/or their activities.

In a specific embodiment, the invention is an in vitro screening method of drug candidates susceptible of preventing and/or treating acne as well as acne associated disorders (e.g., hyperseborrhoea); said method comprising the following steps:

a. Collecting at least two biological samples: one mimics pathological acne lesion condition and the other mimics healthy condition;

b. Contacting at least one sample or a mixture of samples with one or more drug candidates to be tested;

c. Measuring gene expression or gene expression product level or activity in the biological samples or mixture obtained in b);

d. Selecting drug candidates which are capable of modulating gene expression or gene expression product level or activity measured in said samples or mixture obtained in b) and comparing the levels with a control sample, i.e., not mixed with drug candidate.

By “modulate” it is understood any effect on expression or activity of biomarkers/gene expression products, any effect on genes or on activity of at least one of their expression promoter(s) and preferentially any effect inducing e.g. a down regulation or an up regulation, a stimulation, an inhibition, totally or partially.

In the context of the present invention, it is understood that “expression of biomarkers/gene expression product” refers to a quantity of a protein or any else product resulting from the transcription and/or translation of a gene. By activity is meant biological activity.

By “activity of gene promoter(s)” is meant the promoter(s) capacity to trigger DNA sequence transcription under the control of said promoter(s).

In a particular embodiment, in the screening method the gene expression products at step c) are selected from the following list or those mentioned in Table 4:

Beta 4 defensin (DEFB4); skin-derived (SKALP) protease inhibitor 3 (PI3); ligand 2 chemokine (C-X-C motif) (CXCL2); apolipoprotein B mRNA editing enzyme (APOBEC3A); mitochondrial superoxide dismutase 2 (SOD2); granzyme B (GZMB); ligand 2 chemokine (C-X-C motif) (CXCL2); receptor 1 chemokine (C-C motif) (CCR1); leukotriene B4 receptor (LTB4R); tumor necrosis factor receptor superfamily member 1B (TNFRSF1B); complement component 3a receptor 1 (C3AR1); G protein-coupled receptor 65 (GPR65); baculoviral IAP repeat-containing 1 (BIRC1); serum/glucocorticoid regulated kinase (SGK); CD28 antigen (CD28); apoptosis caspase activation inhibitor (AVEN); TNF receptor-associated factor 3 (TRAF3); aldo-keto reductase family 1, member B10 (AKR1B10); phospholipase A2, group IIA (platelets, synovial fluid) (PLA2G2A); UDP glycosyltransferase 1 family, polypeptide A10 (UGT1A10); phospholipid scramblase 1 (PLSCR1); serum amyloid A2 (SAA2); fatty acid binding protein 5 (psoriasis-associated) (FABP5); phosphoinositide-3-kinase, catalytic delta polypeptide (PIK3CD); secretoglobin, family 1D, member 2 (SCGB1D2); secretoglobin, family 2A, member 1 (SCGB2A1); transmembrane 4 superfamily member 3 (TM4SF3); mutS homolog 5 (MSH5); secretoglobin, family 2A, member 2 (SCGB2A2); frizzled-related protein (FRZB); SRY (sex determining region Y)-box 10 (SOX10); keratin 18 (KRT18); lipase hepatic (LIPC); coagulation factor X (F10); fast troponin C2 (TNNC2); KDEL endoplasmic reticulum protein retention receptor 3 (KDELR3); nuclear receptor subfamily 1, group D, member 1 (NR1D1); CD47 antigen (Rh-related antigen, integrin-associated signal transducer) (CD47); Thy1 cell surface antigen (Thy1); selectin P ligand (SELPG); TIMP metallopeptidase inhibitor 1 (TIMP1); chemokine (C-C motif) ligand 21 (CCL21); S100 calcium binding protein A9 (calgranulin B) (S100A9); chemokine (C-C motif) receptor 2 ///chemokine (C-C motif) receptor 2 (CCR2); chemokine (C-C motif) receptor 5 (CCR5); selectin L (lymphocyte adhesion molecule 1) (SELL).

The compounds to be tested are any kind of compounds, from natural or synthetic source. As synthetic compounds they might be chemically synthesized or from chemical compound data bank, with a defined structure or non-characterized or present in a mixture of compounds.

Several technical assays are available for assessing compounds activity modulating above mentioned biomarkers/gene expression products.

According to a first embodiment, biological samples are transfected cells containing reporter gene operably under the control of a promoter (totally or partially) controlling the expression of an above mentioned gene. Therefore step c) above measures the expression of the reporter gene.

The reporter gene may encode an enzyme that with its corresponding substrate, provides colored product(s) such as CAT (chloramphenicol acetyltransferase), GAL (beta galactosidase), or GUS (beta glucuronidase). It might be either luciferase or GFP (Green Fluorescent Protein) gene.

Reporter gene protein dosage or its activity is typically assessed by coloring, fluorometric or chemoluminescence methods.

According to a second embodiment of the invention, biological samples are cells expressing the gene of interest and the step c) above measures the activity of the gene product.

Any kind of cell is suitable for the invention. Cells may endogenously express the said gene like sebocyte. Organs may be suitable for the instant invention, from animal or human origin like preputial gland or sebaceous gland.

Transformed cells by heterologous nucleic acid encoding the gene expression product of interest might either be suitable. Preferably the said nucleic acid is from animal (preferred mammal) or human origin. A large variety of host cells is suitable for the invention and in particular Cos-7, CHO, BHK, 3T3, HEK293 cells. Cells are transiently or permanently transfected by a nucleic acid of interest with a well known by skilled in the art method and for instance calcium phosphate precipitation, DEAE-dextran, liposome, virus, electroporation or microinjection.

In the above described methods, expression levels of a gene of interest or reporter gene are determined according to transcription or translation rates.

By transcription rate is understood, mRNA levels. By translation is meant, protein production rate.

Quantitative or semi-quantitative methods for mRNA of gene of interest detection are well known by one skilled in the art.

Methods based on mRNA hybridation with nucleic probes are typically known (Northern Blot, RT-PCR, RNase protection). It might be advantageous to use detection markers such as fluorescent, radio-labeled, enzymatic agents or other ligands (for example avidine/biotine).

Gene translation rate may also be assessed by immunological assays of gene expression product. To this aim, polyclonal or monoclonal antibodies may be used. Antibodies manufacturing methods are well known to one skilled in the art. For instance, monoclonal antibody might be produced according to Kôhler and Milstein method (Nature (London), 256: 495-497 (1975) or by cloning a nucleic acid expression clone in hybridoma.

Immunological dosages are assessed by solid or homogeny phase, in one or two time frames; with the so-called sandwich method or with competition method.

According to a preferred embodiment of the invention, the antibody is captured on or into solid support such as microplaques, polystyrene plaques or balls or paramagnetic balls.

ELISA dosage, radio-immuno assays or other type of detecting methods may convey to detect antibody/antigen complex.

### Modulators:

The present invention also features the use of identified modulators of acne lesions biomarkers/genes expression products indicated in Tables 1 and 2 and FIG. 1 and particularly inflammatory acne lesions biomarkers for the preparation of compositions preventing and/or treating acne or acne associated disorders.

This Invention also features the cosmetic use of minor acne associated disorders (e.g., hyperseborrhoea, oily skin).

The method of preventing or treating acne and particularly acne lesions, whether regime or regimen, comprises administering to a patient in need a therapeutical effective quantity of modulator.

In a preferred embodiment of the invention, the drug candidates obtained at step d) of the screening method are inhibitors of up-regulated acne lesions biomarkers/genes expression products and inducers of down-regulated acne lesions biomarkers/genes expression products, preferentially the following genes MMPs genes, genes encoding pro-inflammatory cytokines and genes encoding chemokine receptors and preferably are inhibitors of the following biomarkers/gene expression products as defined above and more preferentially those selected from the list in Tables 1, 2 and FIG. 1.

The term “inhibit” refers to a compound that reduces or decrease, restraint, down-regulate, prevent (totally or partially) or suppress, antagonise, stop, block biomarkers/gene expression product activity. By partially, it is meant a reduction of activity of at least 25%, preferred of at least 35%, more preferred of at least 50% and preferentially of at least from 70% to 90%.

Particularly, the modulator might interact and block the active site of the gene expression product like competitive inhibitor.

A preferred inhibitor is active in a solution at a concentration of at least less than 1 μM, preferred less than 0.1 μM, preferentially less 0.01 μM.

The modulator might be an antibody and preferably a monoclonal antibody. Advantageously, the monoclonal antibody is administered to a patient in a sufficient quantity so as the measure a plasmatic concentration from about 0.01 μg/ml to about 100 μg/ml, preferred from about 1 μg/ml to about 5 μg/ml.

Modulator might be either a polypeptide, a DNA or RNA antisens, a si-RNA or a PNA (“Peptide nucleic acid”, i.e., with a polypeptidic chain substituted by purine and pyrimidine bases and having a DNA-like structure for hybridization to this latter)

The present invention also features the use of identified modulators of acne lesions biomarkers/genes expression products and particularly inflammatory acne lesions biomarkers for the preparation of compositions for preventing and/or treating acne or acne associated disorders.

The present invention also features the cosmetic application of identified modulators of acne lesions biomarkers/genes expression products for the treatment of minor acne associated disorders (for example, hyperseborrhoea, oily skin).

Therefore, modulator compounds are formulated into a composition, associated with a pharmaceutically acceptable vehicle. Those compositions are administered by oral, enteral, parenteral or topic route. Preferably, the route of administration is topical.

For oral route, the composition could be in a tablet form, pills, dragees, syrup, suspension, solution, powder, granules, emulsion, microspheres or nanospheres suspensions or lipid or polymeric vesicles compatible with a control release.

For parenteral route, the composition could be in a solution or suspension form for injection or perfusion.

For topical route, the composition is particularly usable for the treatment of skin and mucosa and could be in a form of unguents, creams, milks, ointments, powders, tampons imbibes, solutions, gels, gel-cream, sprays, lotion, emulsions, suspensions or microspheres or nanospheres suspensions or lipid or polymeric vesicles compatible with a control release. The composition might either be in an anhydic or aqueous form or be an emulsion. In a preferred embodiment, composition is in a gel, cream or lotion form.

The subject compositions comprise from 0.001 to 10%, preferably from 0.01 to 5% by weight/total composition weight of modulator compound.

The composition may also comprise inert additives or mixtures thereof, such as

wetting agents;

taste ameliorating agents;

preservatives such as parahydroxybenzoïc acid esters;

stabilizating agents;

humidity regulating agents;

pH regulating agents;

osmotic pressure modulating agents;

emulsifying agents;

UV-A et UV-B filters

antioxidant agents such as alpha-tocopherol, butylhydroxyanisole or butylhydroxytoluene, Super Oxide Dismutase, Ubiquinol or metal chelating agent.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Table 3 and Figure Legends:

### Table 3—Enhanced Expression of Genes Involved in Inflammation and Matrix Remodeling in Acne Lesions:

To validate the micro-array findings, the mRNA levels of 5 genes involved in inflammation were quantified using quantitative real time PCR. Table 3 describes the fold changes in mRNA expression for MMP-1, MMP-3, IL-8, HBD-4 and granzyme B respectively in inflammatory acne lesions and biopsies of normal skin from the same group of patients. The values shown are means with standard errors indicated by the bars.

FIG. 1: Hierarchical Clustering of Genes Involved in Inflammation:

Hierarchical clustering of genes involved in inflammation from 6 patients with skin biopsies taken at the site of the acne lesion (AL) and at a corresponding site of normal skin (NS) from the same patient. Each row is a gene labeled with gene name or accession number and each column is the patient sample. The color in each cell reflects the level of expression of the corresponding gene in the corresponding sample, relative to its mean level of expression in the entire set of biopsy samples. Expression levels greater than the mean are shaded in red and those below the mean are shaded in blue.

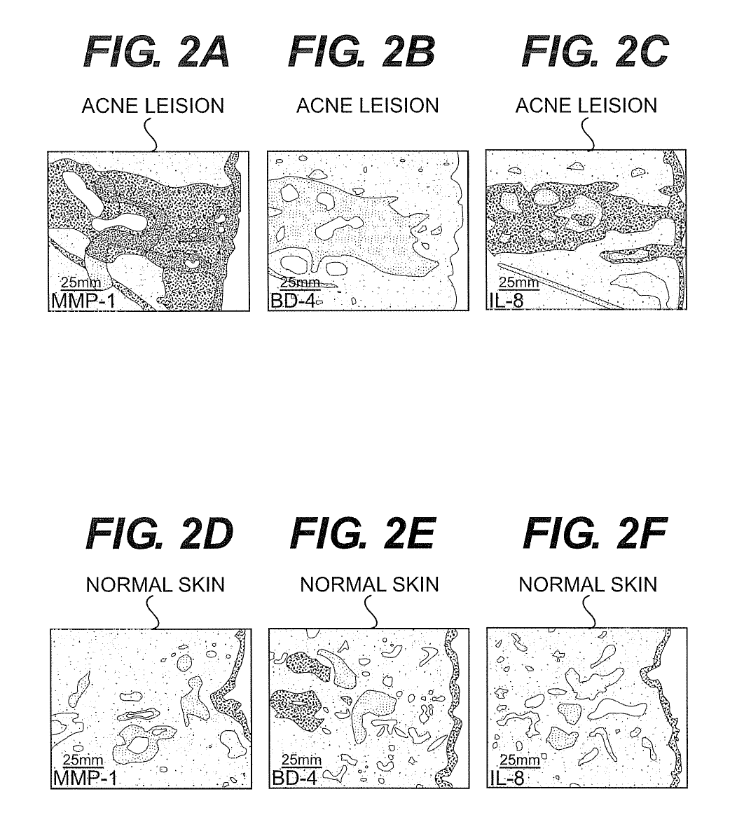

FIG. 2: Confirming Changes in Gene Expression Pattern by Immunohistochemistry:

Immunohistochemistry staining was performed on sections of acne skin and compared to those of clinically normal skin.

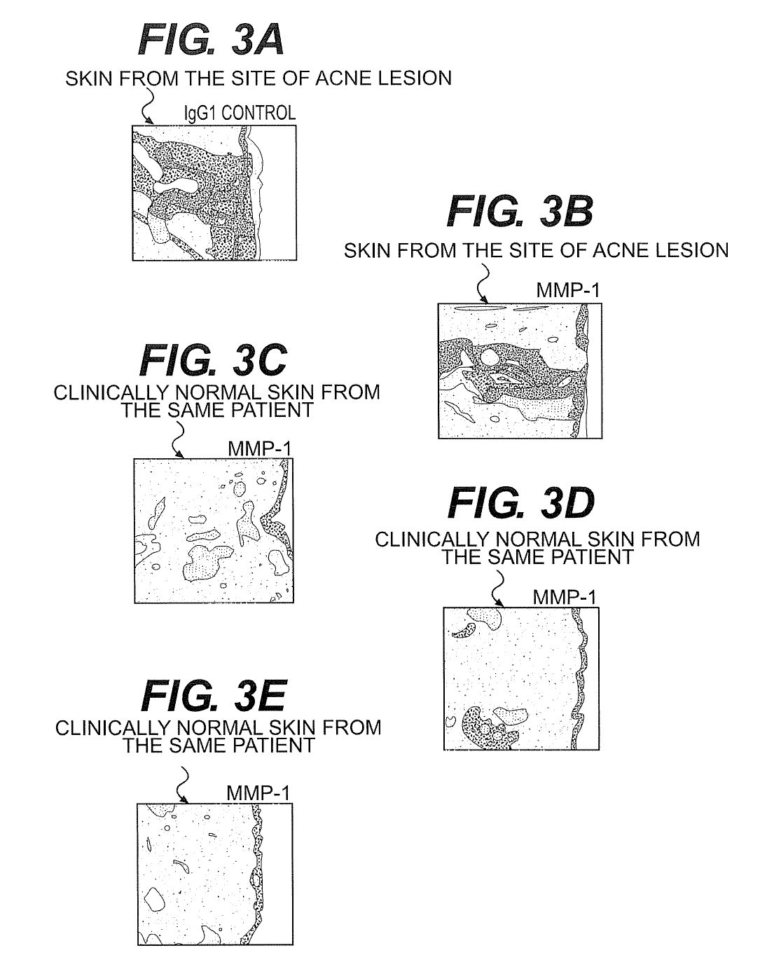

FIG. 3: Immunohistochemistry Staining of MMP-1 in Clinically Normal Skin and Inflammatory Acne:

MMP-1 expressed in the epidermis and sebaceous glands in inflammatory acne shown in (b) compared to IgG1 control shown in (a). Variable levels of MMP-1 staining can be seen in serial sections of clinically normal skin from the same acne patient (c-e). A higher expression level of MMP-1 staining is seen in the epidermis in sections close to microscopically visible perifollicular inflammation (c) and progressively lower levels of MMP-1 staining is seen in sites distal from it (d, e).

### EXAMPLE 1

### Expression of Inflammatory Mediators, Anti-Microbial Peptides and Matrix Metalloproteinases is Increased in Acne Lesions Compared to Uninvolved Skin

### Materials:

The gene chips “HG-U133A 2.0” were purchased from Affymetrix (Santa Clara, Calif.). The MMP-1, MMP-3, IL-8, Human B-defensin 4 (HBD-4) and granzyme B primers for Real Time PCR were obtained from Applied Biosystems (California). The primary antibodies for immunohistochemistry for MMP-1 and IL-8 were purchased from R&D systems (Minneapolis, Minn.) and HBD-4 from Abcam Inc. (Cambridge, Mass.).

### Patient Selection and Tissue Biopsies:

Twelve patients including male and females aged 18 to 45 years were enrolled in the study, 6 patients with acne lesions on the back and 6 subjects without acne. The inclusion criteria for the acne lesion group included: a) males and females aged 18 to 45 years with inflammatory acne on their back, b) subjects without other skin disease in the biopsy area, c) subjects who were willing to have skin biopsies performed from their back and d) subjects that have not been treated with isotretinoin for acne within the previous 6 months. The inclusion criteria for subjects without acne included: a) Males and females aged 18 to 45 years who were willing to have a skin biopsy performed from their back and b) subjects without other skin disease in the biopsy areas. The exclusion criteria (all subjects) included: subjects that were taking oral medications that might influence gene expression in the skin or applying topical medications in the target areas on the back. A punch biopsy (5 mm) of the skin was performed at 2 sites on the back of acne patients, one at the site of inflammatory acne papule and other from a region of clinically normal skin. Normal subjects without acne were subjected to only one biopsy taken from the normal skin on the back.

### Extraction of RNA, Labeling and Hybridization to Probe Arrays:

Skin samples were flash frozen and individually cryosectioned to facilitate RNA isolation. Total RNA was isolated from skin and DNase treated using the RNeasy Fibrous Tissue Kit (Qiagen Inc., Valencia, Calif.) according to the manufacturer's instructions. RNA was ethanol precipitated to concentrate the sample and then quantified using a spectrophotometer. Approximately 2 μg of total RNA from each sample was used to generate double stranded cDNA using a T7-oligo (dT) primer. Biotinylated cRNA, produced through in-vitro transcription, was fragmented and hybridized to an Affymetrix human U133A 2.0 microarray. The arrays were processed on a GeneChip Fluidics Station 450 and scanned on an Affymetrix GeneChip Scanner.

### Quantitative Real-Time PCR:

Quantitative real-time PCR was performed to confirm changes in the level of select genes from the array data. Complimentary DNA was generated from 1 μg of total RNA, primed with oligo dT, using the Superscript First-Strand Synthesis System for RT-PCR (Invitrogen, Carlsbad, Calif.). Assays-on-Demand Taqman Universal PCR Master Mix and primer probe sets (Applied Biosystems) were used to run real-time PCR on ABI's 7900HT Fast Real-Time PCR System with 384-well plate block module (Applied Biosystems, Foster City, Calif.). Samples corresponding to 80 ng total RNA input were run in triplicate for the reference gene TBP as well as 6 genes of interest (MMP-1, MMP-3, DEFB4, IL8, GZMB, and GATA6). No template and no RT controls were also run.

### Immunohistochemistry:

In order to verify changes in gene expression at the protein level, 4 additional subjects with acne were recruited to undergo biopsies of an inflammatory acne lesion and of uninvolved skin from the back. In addition, skin samples from additional subjects without acne were obtained to further assess the expression of the proteins of interest. Immunohistochemistry was performed on these samples following formalin fixation, paraffin embedding and tissue processing as previously described. Sections were incubated with monoclonal antibody to MMP-1 (1:200) and beta-defensin 4 (1:100) and polyclonal antibody to IL-8 (1:50) overnight at 4° C. MMP-1 and IL-8 primary antibodies were purchased from R&D systems while HBD-4 was procured from Abcam. Slides were incubated with biotinylated secondary antibodies against the respective primary antibodies at a dilution of 1:500 for 60-minutes at room temperature followed by 30-minute incubation with the ABC reagent (Vector Labs). The AEC kit from Vector labs was used as the chromogen, which stains as a red color. The sections were counterstained with hematoxylin and slides analyzed by microscopy.

### Clustering:

Out of the 211 genes that were upregulated in acne lesions, 41 were identified as part of the inflammatory probe set generated from the NetAffix analysis center from the Affymetrix website. Hierarchical clustering of patient samples and genes involved in inflammation was performed using the Computer software dChip (Li and Wong. 2003) version 1.3. Patient samples included skin biopsies taken at the site of the acne lesion (AL) and at a site of clinically normal skin (NS) from the same patient. Normalized chip intensity data were imported into dChip. Gene information file for Affymetrix human genome HG-U133A array was obtained from dChip's website at www.dChip.org.

### Statistical Analyses:

Before testing significant changes in gene expression, the expression signals were normalized by using the R-Affy package from Bioconductor (version 1.1, Irizarry et al. 2003a) to remove background noise and non-biological variations among arrays. The background noise was removed from the PM probe intensities using the “RMA” method (Irizarry et al. 2003b), which assumes a global model for the distribution of probe intensities and models the PM probe intensities as the sum of a normal noise component and an exponential signal component. Normalization was done using the quantile normalization method (Bolstad et al. 2003). Quantile normalization assumes that the expression of majority of genes on the arrays does not change in different treatments and the distribution of probe intensities for each array in the dataset is the same. To remove outlier probes and summarize probe intensities within one probe set into a single expression value, “Tukey Biweight” method was applied to the background adjusted, normalized PM intensities. Expression values were obtained based on PM intensities not PM-MM intensities, because PM and MM intensities were found to be highly correlated which suggested that MM intensities composed of background noise as well as probe specific signals. Significant gene expression alterations were identified using computer software Significance Analysis of Microarrays (SAM) (Tusher et al. 2001). SAM assigns a score to each gene on the basis of gene expression change relative to the standard deviation of repeated measurements and identifies genes with statistically significant changes in expression using a permutation procedure. SAM controls the false positives resulting from multiple comparisons through controlling the false discovery rate (FDR) (Benjamini and Hochberg, 1995). FDR is defined as the proportion of false positive genes among all genes that are considered significant.

### Results:

A distinct pattern of gene expression was seen from inflammatory acne lesions compared to uninvolved skin from the same patients. Analysis of the gene expression profiles revealed that 211 genes were upregulated at the site of the acne lesion, while 18 genes were down regulated. A majority of the genes whose expression is increased in acne lesions were involved with the inflammatory processes, and these included a variety of chemokines, anti-microbial peptides, apoptosis inducing proteins and interstitial collagenases (See Table 1 and FIG. 1)

Genes with the greatest fold increases in expression in acne lesions included the matrix metalloproteinases MMP-1 and MMP-3, which had 92 and 64-fold higher expression patterns respectively. Other genes significantly upregulated included the pro-inflammatory cytokines IL-8 (52-fold), and CXCL-2 (16-fold). Significant increases in the expression of chemokine receptor 1 (CCR1), IL-7 receptor, IL-13 receptor and IL-1 family members 5&9 were also noted. These data highlight the prominent role of these cytokines in inflammatory acne, which is in agreement with other studies that demonstrate the role of IL-8 in inducing the recruitment of chemotactic mediators at the site of acne lesions (Vowels et al, 1995).

The expression of several anti-microbial peptides was also significantly increased in acne lesions compared to normal skin from the same patients. These include HBD-4 and granulysin, which were upregulated by more than 33-fold and 2-fold respectively. Genes involved in apoptosis and immune pathways were also significantly upregulated in acne lesions. These included granzyme B that is responsible for target cell lysis in cell-mediated immune responses and GPR65 that is involved with the differentiation of T-cells as well as their apoptosis. Granzyme B was upregulated more than 10-fold in acne lesions when compared to normal skin while that of GPR65 was increased by approximately 2.7-fold. Tables 1 and 2 provide a more comprehensive list of the genes with statistically significant changes in expression from inflammatory acne lesions and normal skin from the same patients.

While very few genes were downregulated in acne lesions compared to normal skin, some important genes regulating key pathways were identified (Table 2). The frizzled related proteins which function as part of the ‘wnt’ signaling pathway were down-regulated by approximately 2-fold. Inhibition of wnt signaling in the skin has been shown to influence stem cell fate in favor of development of sebaceous rather than hair (Merrill et al, 2001). Also downregulated in acne lesion were three genes of the secretoglobin family: secretoglobin family 1D member 2 and secretoglobin family 2A members 1 & 2. Secretoglobin family 1D member 2 and secretoglobin family 2A member 1 are transcriptionally regulated by steroids and bind to androgens and other steroids. The function of secretoglobin family 2A member 2 is unclear.

Gene expression profiles from biopsy samples of normal skin from 6 subjects without acne were compared to profiles obtained from normal skin (uninvolved sites) of 6 patients with acne. No significant changes in gene expression patterns were noted in this analysis. A subset analysis of target genes was performed and again revealed no significant differences in gene expression from normal skin from subjects with and without acne.

### EXAMPLE 2

### Clustering

Using the computer software dChip (Li and Wong. 2003), hierarchical clustering was conducted of the entire set of genes (229) that were significantly upregulated or downregulated from the micro-array data and found that the biopsy samples from inflammatory acne involved skin lesions clustered into a separate group from the uninvolved clinically normal skin from the same group of patients. Using the NetAffix analysis center from the affymetrix website 41 genes were identified from a total of 211 genes upregulated in inflammatory acne to be involved in inflammation. FIG. 1 is a cluster diagram of the inflammatory genes and shows the unique clustering pattern of the samples into 2 groups, one corresponding to involved skin from acne lesion and another corresponding to clinically uninvolved skin form the same group of 6 patients.

### EXAMPLE 3

### qPCR Confirms Gene Array Expression Data of Select Genes

5 Genes of interest were selected based on their fold changes and involvement in inflammation from 5 acne subjects to validate the micro-array findings using qPCR. These genes include, MMP-1, MMP-3, IL-8, β defensin 4 and Granzyme B. QPCR results were normalized to the internal control gene, TATA binding protein (TBP). A robust increase in mRNA expression was seen for all 5 genes tested with the magnitude of the fold change greater than that observed with the microarray (Table 3XX Nishit please include also the fold changes from microarray in this table).

### EXAMPLE 4

### Immunohistochemistry Demonstrates Tissue Localization of Select Proteins in Inflammatory Acne Lesions and Normal Skin

To further confirm the differences in tissue expression and distribution patterns of MMP-1 (interstitial collagenase), IL-8 (pro-inflammatory cytokine) and HBD-4 (an anti-microbial peptide), immunohistochemistry was conducted on inflammatory acne lesions, uninvolved skin from acne subjects and on normal skin from subjects without acne. Increased expression of all 3 proteins was noted in inflammatory acne lesions when compared to normal skin from the acne subjects (FIG. 2). The expression of HBD-4 was greater in the epidermis of inflammatory acne lesions when compared to the epidermis in the uninvolved skin (FIGS. 3 A&C). Immunoreactivity with antibody to IL-8 was noted at the follicular and perifollicular sites of the inflammation in the acne lesion (FIG. 2B). IL-8 immunoreactivity was relatively absent in the normal skin (FIG. 2D).

While high levels of MMP-1 expression were found in the epidermis and sebaceous glands in biopsy sections from the inflammatory acne lesions (FIG. 3B); significant variation in MMP-1 expression was found in skin biopsies from the uninvolved skin from the same patient (FIG. 3C, D, E). Performing serial sectioning of clinically normal skin of a patient with acne, perifollicular inflammation was observed, suggestive of an early clinically inapparent acne lesion. MMP-1 immunoreactivity was increased in areas that were close to perifollicular inflammation, while very little MMP-1 immunoreactivity was observed in those areas that had no microscopically visible inflammation. The higher levels of MMP-1 expression at some sites of normal skin need to further examined to determine if changes in MMP-1 expression are amongst the earliest changes prior to the development of comedones and inflammatory acne at that site.

### DISCUSSION

Acne is recognized as a chronic inflammatory disease, characterized by increased sebum production, abnormal follicular differentiation, and bacterial colonization. Though significant advances have been made in the last decade in identifying the pathophysiological mechanisms involved in acne, there are no published micro-array studies analyzing the differential pattern of gene expression in inflammatory acne lesions and normal skin. This invention is the first study to provide such a comprehensive comparison. In this regard, 211 genes were significantly upregulated in inflammatory acne lesions, many of which, as expected, are involved in inflammation. The major genes whose expression was increased have been implicated in acne. These include matrix metalloproteinases, β-defensin 4, IL-8, and granulysin. In contrast, a much smaller set of genes (18) was downregulated, 3 of which are in the secretoglobin family.

The recognition of microbial pathogens by the cells of the immune system triggers host defense mechanisms to combat infection. These include anti-microbial peptides, inflammatory cytokines and pro-apoptotic enzymes. However, activation of these same mechanisms also results in tissue injury and scarring, a feature commonly observed in inflammatory acne. The data generated in this study supports many of the recent findings regarding inflammatory mediators in acne. For example, the extent to which P. acnes induces the expression of the anti-microbial peptide, β-defensin 2 (now known as β-defensin 4)) and IL-8 (Nagy, et al., 2005). Since P. acnes is routinely present in the skin of most individuals, and no correlation from the number of bacteria and the severity and type of acne has been found, it is possible that strain to strain variations in the expression of β-defensin 4 or IL-8 might play a role in the development of inflammatory acne. β-defensin 4 is as an important member of the defensin family of anti-microbial peptides and has strong anti-microbial activity against both gram-positive and gram negative bacteria. The present data demonstrate that β-defensin 4 is expressed in the epidermis of inflammatory acne lesions, but not in normal skin and these results are in agreement with other studies demonstrating an increase in expression of p defensin 1 and 2 in the epidermis of the inflammatory acne lesions (Chronnell, et al., 2001).

Granulysin is another important anti-microbial peptide whose expression is significantly increased in inflammatory acne lesions in the present study. In recent years granulysin has gained importance as a peptide that can perform the dual function of being a cytotoxic agent against pathogenic bacteria as well as a pro-inflammatory agent that functions as a chemo attractant and activates monocytes to produce cytokines (Deng, et al., 2005). Antibiotics to combat P. acnes and other bacteria have been commonly used in the treatment of acne and thus the upregulation of these anti-microbial peptides produced by the body can be helpful in killing the bacteria. However recent studies have shown that while some chemokines are reduced as a consequence of the microbicidal effect of granulysin peptides, the levels of IL-8 remain unchanged (McInturff, et al., 2005).

Interleukin-8, a prototypic human chemokine has been discovered since 1990 as the founding member of the chemokine family. IL-8 is a major mediatory of inflammatory response and a strong chemotactic factor for neutrophis, basophils and T-cells (Zachariae, 1993). The present study demonstrates that it is markedly upregulated in inflammatory acne lesions. The activation of this chemokine is regulated by a combination of 3 major mechanisms, namely, a) the transcriptional activation by the NF-κB and JUN protein kinase pathways, b) stabilization of the mRNA by the p38 MAPK pathway and c) derepression of the gene promotor (Harant et al, 1996; Hoffmann et al, 2002; Mukaida et al, 1994). As in several inflammatory diseases, IL-8 has been implicated to be playing a major role in mounting an inflammatory response in acne lesions. Several studies have focused on the role of I bacteria in inducing IL-8 and discovered that this process may be mediated by the activation of transcription factor NF-kB (Chen et al, 2002; Vowels, et al., 1995). Thus a detailed understanding of the pathways governing IL-8 production may help to identify newer targets and better therapeutics for acne treatment.

The more recent studies by Kang et al have focused on identifying the various intra-cellular signaling cascades associated with transcription factors involved in inflammation and matrix degradation in acne lesions. The activation of NF-κB pathway as evidenced by nuclear localization of p65 and p50, and the subsequent regulation of inflammatory cytokines like TNF-α, IL-1β, and IL-8 have provided clues that help the understanding of the molecular pathways governing inflammation in acne (Kang et al, 2005). The present results are in agreement with studies by Kang et al. in demonstrating increased expression of matrix metalloproteinases like MMP-1 and MMP-3 and cytokines especially IL-8 in the inflammatory acne lesion. Because MMP-1 expression was also observed in some areas of clinically normal skin, which had microscopically visible areas of inflammation, one hypothesizes that MMP-1 could play a role during the initial stages of inflammation and that inflammation may be one of the preceding events to the clinically visible acne lesions as previously suggested (Jeremy, et al., 2003).

The present study has also brought to focus the strong upregulation of granzyme B an essential component of the apoptotic pathway that is necessary for target cell lysis in cell-mediated immune response (Heibein et al, 2000). Granzyme B is commonly found in granules produced by cytolytic T lymphocytes (CTLs) and natural killer (NK) cells. CTLs and NK cells use perforin and granzyme B containing granules to destroy cells infected with intracellular pathogens (Trapani and Smyth, 2002). Granzyme B can induce target cell death via two complementary pathways, a cytosolic pathway involving cascade activation of caspases and a nuclear pathway involving CDC-2 activation (Talanian et al, 1997). While very little is known about the role played by granzyme B in the pathogenesis of acne, the strong upregulation of granzyme B in acne lesions as observed in the present studies will help foster interest into a more in depth study of this protease in acne.

These results strongly bring to light the role of the various proteins involved in inflammation and matrix remodeling in inflammatory acne lesions and provides a more detailed look at the differential patterns of gene expression in an inflammatory acne and clinically normal skin from the same group of patients. The upregulation of anti-microbial peptides is of interest further supporting their role in the endogenous inflammatory response to bacterial pathogens and perhaps also providing a rationale for their potential therapeutic use in acne. Critical questions remain however as to the nature of the initiating events in the development of acne lesions. It is likely that the profiles of gene expression in any inflammatory process in the skin are quite similar and that many of the changes observed in inflammatory lesions are likely to be secondary to as of yet unidentified primary pathogenic events. The challenge lies ahead in identifying these primary events in acne as well as in other inflammatory diseases.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

### REFERENCES

Braff M H, Bardan A, Nizet V, Gallo R L: Cutaneous defense mechanisms by antimicrobial peptides. J Invest Dermatol., 125: 9-13, 2005.
Chen Q, Koga T, Uchi H, et al: Propionibacterium acnes-induced IL-8 production may be mediated by NF-kappaB activation in human monocytes. J Dermatol Sci., 29: 97-103, 2002.
Chronnell C M, Ghali L R, Ali R S, et al: Human beta defensin-1 and -2 expression in human pilosebaceous units: upregulation in acne vulgaris lesions. J Invest Dermatol., 117: 1120-5, 2001.
De Young L M, Young J M, Ballaron S J, Spires D A, Puhvel S M: Intradermal injection of Propionibacterium acnes: a model of inflammation relevant to acne. J Invest Dermatol., 83: 394-8, 1984.
Deng A, Chen S, Li Q, Lyu S C, Clayberger C, Krensky A M: Granulysin, a cytolytic molecule, is also a chemoattractant and proinflammatory activator. J. Immunol., 174: 5243-8, 2005.
Freedberg I M, Tomic-Canic M, Komine M, Blumenberg M: Keratins and the keratinocyte activation cycle. J Invest Dermatoll., 116: 633-40, 2001.
Harant H, de Martin R, Andrew P J, Foglar E, Dittrich C, Lindley I J: Synergistic activation of interleukin-8 gene transcription by all-trans-retinoic acid and tumor necrosis factor-alpha involves the transcription factor NF-kappaB. J Biol Chem., 271: 26954-61, 1996.
Harder J, Meyer-Hoffert U, Wehkamp K, Schwichtenberg L, Schroder J M: Differential gene induction of human beta-defensins (hBD-1, -2, -3, and -4) in keratinocytes is inhibited by retinoic acid. J Invest Dermatol., 123: 522-9, 2004.
Heibein J A, Goping I S, Barry M, Pinkoski M J, Shore G C, Green D R, Bleackley R C: Granzyme B-mediated cytochrome c release is regulated by the Bcl-2 family members bid and Bax. J Exp Med., 192: 1391-402, 2000.
Hoffmann E, Dittrich-Breiholz O, Holtmann H, Kracht M: Multiple control of interleukin-8 gene expression. J Leukoc Biol., 72: 847-55, 2002.
Jappe U, Ingham E, Henwood J, Holland K T: Propionibacterium acnes and inflammation in acne; P. acnes has T-cell mitogenic activity. Br J Dermatol., 146: 202-9, 2002.
Jeremy A H, Holland D B, Roberts S G, Thomson K F, Cunliffe W J: Inflammatory events are involved in acne lesion initiation. J Invest Dermatol., 121: 20-7, 2003.
Kang S, Cho S, Chung J H, Hammerberg C, Fisher G J, Voorhees J J: Inflammation and extracellular matrix degradation mediated by activated transcription factors nuclear factor-kappaB and activator protein-1 in inflammatory acne lesions in vivo. Am J Pathol., 166: 1691-9, 2005.
Kim J, Ochoa M T, Krutzik S R, et al: Activation of toll-like receptor 2 in acne triggers inflammatory cytokine responses. J Immunol., 169: 1535-41, 2002.
McInturff J E, Wang S J, Machleidt T, et al: Granulysin-derived peptides demonstrate antimicrobial and anti-inflammatory effects against Propionibacterium acnes. J Invest Dermatol., 125: 256-63, 2005.
Merrill B J, Gat U, DasGupta R, Fuchs E: Tcf3 and Lef1 regulate lineage differentiation of multipotent stem cells in skin. Genes Dev., 15: 1688-705, 2001.
Mukaida N, Okamoto S, Ishikawa Y, Matsushima K: Molecular mechanism of interleukin-8 gene expression. J Leukoc Biol., 56: 554-8, 1994.
Nagy I, Pivarcsi A, Koreck A, Szell M, Urban E, Kemeny L: Distinct strains of Propionibacterium acnes induce selective human beta-defensin-2 and interleukin-8 expression in human keratinocytes through toll-like receptors. J Invest Dermatol., 124: 931-8, 2005.
Schaller M, Loewenstein M, Borelli C, Jacob K, Vogeser M, Burgdorf W H, Plewig G: Induction of a chemoattractive proinflammatory cytokine response after stimulation of keratinocytes with Propionibacterium acnes and coproporphyrin III. Br J Dermatol., 153: 66-71, 2005.
Schroder J M: [Epithelial antimicrobial peptides: local innate defense effector molecules]. Ann Dermatol Venereol., 131: 411-6, 2004.
Selsted M E, Ouellette A J: Mammalian defensins in the antimicrobial immune response. Nat Immunol., 6: 551-7, 2005.
Talanian R V, Yang X, Turbov J, et al: Granule-mediated killing: pathways for granzyme B-initiated apoptosis. J Exp Med., 186: 1323-31, 1997.
Trapani J A, Smyth M J: Functional significance of the perforin/granzyme cell death pathway. Nat Rev Immunol., 2: 735-47, 2002.
Vowels B R, Yang S, Leyden J J: Induction of proinflammatory cytokines by a soluble factor of Propionibacterium acnes: implications for chronic inflammatory acne. Infect Immun., 63: 3158-65, 1995.
Zachariae C O: Chemotactic cytokines and inflammation. Biological properties of the lymphocyte and monocyte chemotactic factors ELCF, MCAF and IL-8. Acta Derm Venereol., Suppl (Stockh) 181: 1-37, 1993.

TABLE 1
List of genes up regulated in acne lesions when compared to normal skin

| | | | | |
|---|---|---|---|---|
| | | Fold | | |
| Probe Set ID | Accession | Change | Gene Title | Gene Symbol |

List of Genes upregulated by more than 10 fold in acne lesions

| | | | | |
|---|---|---|---|---|
| 204475_at | NM_002421 | 92.166 | matrix metalloproteinase 1 | MMP1 |
| 205828_at | NM_002422 | 64.020 | matrix metalloproteinase 3 | MMP3 |
| 202859_x_at | NM_000584 | 52.521 | interleukin 8 | IL8 |
| 207356_at | NM_004942 | 33.300 | defensin, beta 4 | DEFB4 |
| 203691_at | NM_002638 | 19.354 | protease inhibitor 3, skin-derived (SKALP) | PI3 |
| 41469_at | L10343 | 17.495 | protease inhibitor 3, skin-derived (SKALP) | PI3 |
| 204470_at | NM_001511 | 16.152 | chemokine (C—X—C motif) ligand 2 | CXCL2 |
| 210873_x_at | U03891 | 15.921 | apolipoprotein B mRNA editing enzyme | APOBEC3A |
| 216841_s_at | X15132 | 11.529 | superoxide dismutase 2, mitochondrial | SOD2 |
| 210164_at | J03189 | 10.630 | granzyme B | GZMB |

Upregulation of genes involved in the inflammatory pathway (in acne lesions)

| | | | | |
|---|---|---|---|---|
| 202859_x_at | NM_000584 | 52.521 | interleukin 8 | IL8 |
| 204470_at | NM_001511 | 16.152 | chemokine (C—X—C motif) ligand 2 | CXCL2 |
| 203535_at | NM_002965 | 5.754 | S100 calcium binding protein A9 (calgranulin B) | S100A9 |
| 205098_at | AI421071 | 4.057 | chemokine (C-C motif) receptor 1 | CCR1 |
| 205099_s_at | AI421071 | 3.126 | chemokine (C-C motif) receptor 1 | CCR1 |
| 219403_s_at | AF155510 | 2.920 | heparanase | HPSE |
| 208607_s_at | NM_030754 | 2.659 | serum amyloid A2 | SAA2 |
| 216388_s_at | U33448 | 2.308 | leukotriene B4 receptor | LTB4R |
| 203508_at | NM_001066 | 2.287 | tumor necrosis factor receptor superfamily member 1B | TNFRSF1B |
| 209906_at | U62027 | 2.238 | complement component 3a receptor 1 | C3AR1 |

Upregulation of genes involved in the apoptotic pathway (in acne lesions)

| | | | | |
|---|---|---|---|---|
| 210164_at | J03189 | 10.630 | granzyme B | GZMB |
| 214467_at | NM_003608 | 2.772 | G protein-coupled receptor 65 | GPR65 |
| 204860_s_at | AI817801 | 2.272 | baculoviral IAP repeat-containing 1 | BIRC1 |
| 201743_at | NM_000591 | 2.211 | CD14 antigen | CD14 |
| 201739_at | NM_005627 | 1.663 | serum/glucocorticoid regulated kinase | SGK |
| 206545_at | NM_006139 | 1.658 | CD28 antigen (Tp44) | CD28 |
| 219366_at | NM_020371 | 1.627 | apoptosis, caspase activation inhibitor | AVEN |
| 208315_x_at | NM_003300 | 1.393 | TNF receptor-associated factor 3 | TRAF3 |

Upregulation of genes involved in lipid and steroid metabolism pathway (in acne lesions)

| | | | | |
|---|---|---|---|---|
| 206561_s_at | NM_020299 | 8.389 | aldo-keto reductase family 1, member B10 | AKR1B10 |
| 203649_s_at | NM_000300 | 5.518 | phospholipase A2, group IIA (platelets, synovial fluid) | PLA2G2A |
| 215125_s_at | AV691323 | 2.804 | UDP glycosyltransferase 1 family, polypeptide A10 | UGT1A10 |
| 202430_s_at | NM_021105 | 2.959 | phospholipid scramblase 1 | PLSCR1 |
| 208607_s_at | NM_030754 | 2.659 | serum amyloid A2 | SAA2 |
| 202345_s_at | NM_001444 | 2.268 | fatty acid binding proteins (psoriasis-associated) | FABP5 |
| 204446_s_at | NM_000698 | 2.128 | arachidonate 5-lipoxygenase | ALOX5 |
| 203879_at | U86453 | 1.461 | phosphoinositide-3-kinase, catalytic, delta polypeptide | PIK3CD |

TABLE 2
List of genes down regulated in acne lesions compared to normal skin

| | | | | |
|---|---|---|---|---|
| | | Fold | | |
| Probe Set ID | Accession | change | Gene Title | Gene Symbol |

Complete list of genes that were down regulated in acne lesions

| | | | | |
|---|---|---|---|---|
| 206799_at | NM_006551 | −2.889 | secretoglobin, family 1D, member 2 | SCGB1D2 |
| 205979_at | NM_002407 | −2.883 | secretoglobin, family 2A, member 1 | SCGB2A1 |
| 203824_at | NM_004616 | −2.677 | transmembrane 4 superfamily member 3 | TM4SF3 |
| 212913_at | BE674960 | −2.586 | mutS homolog 5 (E. coli) | MSH5 |
| 206378_at | NM_002411 | −2.227 | secretoglobin, family 2A, member 2 | SCGB2A2 |
| 221406_s_at | NM_025259 | −1.991 | mutS homolog 5 (E. coli) | MSH5 |
| 203697_at | U91903 | −1.939 | frizzled-related protein | FRZB |
| 219127_at | NM_024320 | −1.879 | hypothetical protein MGC11242 | MGC11242 |
| 209842_at | AI367319 | −1.766 | SRY (sex determining region Y)-box 10 | SOX10 |
| 201596_x_at | NM_000224 | −1.656 | keratin 18 | KRT18 |
| 206606_at | NM_000236 | −1.571 | lipase, hepatic | LIPC |
| 205620_at | NM_000504 | −1.547 | coagulation factor X | F10 |
| 219717_at | NM_017741 | −1.522 | hypothetical protein FLJ20280 | FLJ20280 |
| 205388_at | NM_003279 | −1.509 | troponin C2, fast | TNNC2 |
| 207265_s_at | NM_016657 | −1.442 | KDEL endoplasmic reticulum protein retention receptor 3 | KDELR3 |
| 206747_at | NM_014696 | −1.328 | KIAA0514 | KIAA0514 |
| 210006_at | BC002571 | −1.325 | DKFZP564O243 protein | DKFZP564O243 |
| 31637_s_at | X72631 | −1.310 | nuclear receptor subfamily 1, group D, member 1 | NR1D1 |

TABLE 3
Enhanced expression of genes involved in
inflammation and matrix remodeling in acne lesions

Fold Change in mRNA
expression in biopsy
samples from inflammatory
acne lesions compared to
clinically normal skin

| | Mean Value | Std Error |
|---|---|---|
| MMP-1 | +170 | ±18 |
| MMP-3 | +165 | ±32 |
| IL-8 | +935 | ±338 |
| HBD-4 | +167 | ±20 |
| Granzyme B | +16 | ±2 |

TABLE 4
list of genes and biomarkers/gene expression products for modulators screening

| | | Gene |
|---|---|---|
| Accession | Gene title | symbol |
| NM_004942 | defensin, beta 4 | DEFB4 |
| NM_002638 | protease inhibitor 3, skin-derived (SKALP) | PI3 |
| NM_001511 | chemokine (C—X—C motif) ligand 2 | CXCL2 |
| U03891 | apolipoprotein B mRNA editing enzyme | APOBEC3A |
| X15132 | superoxide dismutase 2, mitochondrial | SOD2 |
| J03189 | granzyme B | GZMB |
| NM_001511 | chemokine (C—X—C motif) ligand 2 | CXCL2 |
| AI421071 | chemokine (C-C motif) receptor 1 | CCR1 |
| U33448 | leukotriene B4 receptor | LTB4R |
| NM_001066 | tumor necrosis factor receptor superfamily member 1B | TNFRSF1B |
| U62027 | complement component 3a receptor 1 | C3AR1 |
| NM_003608 | G protein-coupled receptor 65 | GPR65 |
| AI817801 | baculoviral IAP repeat-containing 1 | BIRC1 |
| NM_005627 | serum/glucocorticoid regulated kinase | SGK |
| NM_006139 | CD28 antigen (Tp44) | CD28 |
| NM_020371 | apoptosis, caspase activation inhibitor | AVEN |
| NM_003300 | TNF receptor-associated factor 3 | TRAF3 |
| NM_020299 | aldo-keto reductase family 1, member B10 | AKR1B10 |
| NM_000300 | phospholipase A2, group IIA (platelets, synovial fluid) | PLA2G2A |
| AV691323 | UDP glycosyltransferase 1 family, polypeptide A10 | UGT1A10 |
| NM_021105 | phospholipid scramblase 1 | PLSCR1 |
| NM_030754 | serum amyloid A2 | SAA2 |
| NM_001444 | fatty acid binding protein 5 (psoriasis-associated) | FABP5 |
| U86453 | phosphoinositide-3-kinase, catalytic, delta polypeptide | PIK3CD |
| NM_006551 | secretoglobin, family 1D, member 2 | SCGB1D2 |
| NM_002407 | secretoglobin, family 2A, member 1 | SCGB2A1 |
| NM_004616 | transmembrane 4 superfamily member 3 | TM4SF3 |
| BE674960 | mutS homolog 5 (E. coli) | MSH5 |
| NM_002411 | secretoglobin, family 2A, member 2 | SCGB2A2 |
| U91903 | frizzled-related protein | FRZB |
| AI367319 | SRY (sex determining region Y)-box 10 | SOX10 |
| NM_000224 | keratin 18 | KRT18 |
| NM_000236 | lipase, hepatic | LIPC |
| NM_000504 | coagulation factor X | F10 |
| NM_003279 | troponin C2, fast | TNNC2 |
| NM_016657 | KDEL endoplasmic reticulum protein retention receptor 3 | KDELR3 |
| X72631 | nuclear receptor subfamily 1, group D, member 1 | NR1D1 |
| Z25521 | CD47 antigen (Rh-related antigen, integrin-associated signal | CD47 |
| | transducer) | |
| NM_006288 | Thy1 cell surface antigen | Thy1 |
| NM_003006 | selectin P ligand | SELPG |
| NM_003254 | TIMP metallopeptidase inhibitor 1 | TIMP1 |
| NM_002989 | chemokine (C-C motif) ligand 21 | CCL21 |
| NM_002965 | S100 calcium binding protein A9 (calgranulin B) | S100A9 |
| NM_000647 | chemokine (C-C motif) receptor 2 /// chemokine (C-C motif) | CCR2 |
| | receptor 2 | |
| NM_000579 | chemokine (C-C motif) receptor 5 | CCR5 |
| NM_000655 | selectin L (lymphocyte adhesion molecule 1) | SELL |

What is claimed is:

1. A method of in vitro screening a drug candidate, the method comprising determining the capacity of the candidate to modulate expression of a selected gene or activity of the selected gene expression product wherein the selected gene or gene expression product is an acne lesion biomarker or gene expression product selected from the group consisting of matrix metalloproteinase 1 (MMP1); matrix metalloproteinase 3 (MMP3); interleukin 8 (IL8); beta 4defensin (DEFB4); skin-derived (SKALP) protease inhibitor 3 (PI3); chemokine (C-X-C motif) ligand 2 (CXCL2); apolipoprotein B mRNA editing enzyme (APOBEC3A); superoxide dismutase 2, mitochondrial (SOD2); granzyme B (GZMB); S100 calcium binding protein A9 (calgranulin B) (S100A9); chemokine (C-C motif) receptor 1 (CCR1); heparanase (HPSE); serum amyloid A2 (SAA2); leukotriene B4 receptor (LTB4R); tumor necrosis factor receptor superfamily member 1B (TNFRSF1B); complement component 3a receptor 1 (C3AR1); G protein-coupled receptor 65 (GPR65); baculoviral IAP repeat-containing 1 (BIRC1); CD14 antigen (CD14); serum/glucocorticoid regulated kinase (SGK); CD28 antigen (Tp44) (CD28); apoptosis caspase activation inhibitor (AVEN); TNF receptor-associated factor 3 (TRAF3); aldo-keto reductase family 1, member B10 (AKR1B10); phospholipase A2, group IIA (platelets, synovial fluid) (PLA2G2A); UDP glycosyltransferase 1 family, polypeptide A10 (UGT A10); phospholipid scramblase 1 (PLSCR1); serum amyloid A2 (SAA2); fatty acid binding protein 5 (psoriasis-associated) (FABP5); arachidonate 5-lipoxygenase (ALOX5); phosphoinositide-3-kinase, catalytic, delta polypeptide (PIK3CD); secretoglobin, family 1D, member 2 (SCGB1D2); secretoglobin, family 2A, member 1 (SCGB2A1); transmembrane 4 superfamily member 3 (TM4SF3); mutS homolog 5 (E. coli) (MSH5); secretoglobin, family 2A, member 2 (SCGB2A2); mutS homolog 5 (E. coli) (MSH5); frizzled-related protein (FRZB); hypothetical protein MGC11242 (MGC11242); SRY (sex determining region Y)-box 10 (SOX10); keratin 18 (KRT18); lipase hepatic (LIPC); coagulation factor X (F10); hypothetical protein FLJ20280 (FLJ20280); fast troponin C2, (TNNC2); KDEL endoplasmic reticulum protein retention receptor 3 (KDELR3); KIAA0514; DKFZP5640243 protein; nuclear receptor subfamily 1, group D, member 1 (NR1D1); CD47 antigen (Rh-related antigen, integrin-associated signal transducer) (CD47); Thy1 cell surface antigen (Thy1); selectin P ligand (SELPG); TIMP metallopeptidase inhibitor 1 (TIMP1); chemokine (C-C motif) ligand 21 (CCL21); S100 calcium binding protein A9 (calgranulin B) (S100A9); chemokine (C-C motif) receptor 2 ///chemokine (C-C motif) receptor 2 (CCR2); chemokine (C-C motif) receptor 5 (CCR5); and selectin L (lymphocyte adhesion molecule 1) (SELL).

2. The method as defined by claim 1, wherein the biomarker is an inflammatory acne lesion biomarker.

3. The method as defined by claim 1, wherein the biomarker is a biological target.

4. The method as defined by claim 1, further comprising:
a) collecting at least two biological samples; wherein a first sample mimics an acne lesion, and a second sample mimics a healthy condition;
b) contacting at least one sample or a mixture of samples with one or more drug candidates to be tested;
c) measuring gene expression or gene expression product level or activity in the biological samples or mixture obtained in b); and
d) selecting drug candidates which are capable of modulating gene expression or gene expression product level or activity measured in the samples or mixture obtained in b) and comparing the levels with a sample not mixed with the drug candidate.

5. The method as defined by claim 5, wherein the gene expression product at step c) is selected from the group consisting of beta 4 defensin (DEFB4); skin-derived (SKALP) protease inhibitor 3 (PI3); ligand 2 chemokine (C-X-C motif) (CXCL2); apolipoprotein B mRNA editing enzyme (APOBEC3A); mitochondrial superoxide dismutase 2 (SOD2); granzyme B (GZMB); ligand 2 chemokine (C-X-C motif) (CXCL2); receptor 1 chemokine (C-C motif) (CCR1); leukotriene B4 receptor (LTB4R); tumor necrosis factor receptor superfamily member 1B (TNFRSF1B); complement component 3a receptor 1 (C3AR1); G protein-coupled receptor 65 (GPR65); baculoviral IAP repeat-containing 1 (BIRC1); serum/glucocorticoid regulated kinase (SGK); CD28 antigen (CD28); apoptosis caspase activation inhibitor (AVEN); TNF receptor-associated factor 3 (TRAF3); aldo-keto reductase family 1, member B10 (AKR1B10); phospholipase A2, group IIA (platelets, synovial fluid) (PLA2G2A); UDP glycosyltransferase 1 family, polypeptide A10 (UGT1A10); phospholipid scramblase 1 (PLSCR1); serum amyloid A2 (SAA2); fatty acid binding protein 5 (psoriasis-associated) (FABP5); phosphoinositide-3-kinase, catalytic delta polypeptide (PIK3CD); secretoglobin, family 1D, member 2 (SCGB1D2); secretoglobin, family 2A, member 1 (SCGB2A1); transmembrane 4 superfamily member 3 (TM4SF3); mutS homolog 5 (MSH5); secretoglobin, family 2A, member 2 (SCGB2A2); frizzled-related protein (FRZB); SRY (sex determining region Y)-box 10 (SOX10); keratin 18 (KRT18); lipase hepatic (LIPC); coagulation factor X (F10); fast troponin C2 (TNNC2); KDEL endoplasmic reticulum protein retention receptor 3 (KDELR3); nuclear receptor subfamily 1, group D, member 1 (NR1D1); CD47 antigen (Rh-related antigen, integrin-associated signal transducer) (CD47); Thy1 cell surface antigen (Thy1); selectin P ligand (SELPG); TIMP metallopeptidase inhibitor 1 (TIMP1); chemokine (C-C motif) ligand 21 (CCL21); S100 calcium binding protein A9 (calgranulin B) (S100A9); chemokine (C-C motif) receptor 2 ///chemokine (C-C motif) receptor 2 (CCR2); chemokine (C-C motif) receptor 5 (CCR5); and selectin L (lymphocyte adhesion molecule 1) (SELL).

6. The method as defined by claim 5, wherein the drug candidates at step d) are inhibitors of up-regulated gene expression product.

7. The method as defined by claim 6, wherein the drug candidates of step d) are MMPs genes, genes encoding pro-inflammatory cytokines and genes encoding chemokine receptors.

8. The method as defined by claim 4, wherein the drug candidates at step d) are inhibitors of biomarkers selected from the group consisting of matrix metalloproteinase 1 (MMP1); matrix metalloproteinase 3 (MMP3); interleukin 8 (IL8); beta 4defensin (DEFB4); skin-derived (SKALP) protease inhibitor 3 (PI3); chemokine (C-X-C motif) ligand 2 (CXCL2); apolipoprotein B mRNA editing enzyme (APOBEC3A); superoxide dismutase 2, mitochondrial (SOD2); granzyme B (GZMB); S100 calcium binding protein A9 (calgranulin B) (S100A9); chemokine (C-C motif) receptor 1 (CCR1); heparanase (HPSE); serum amyloid A2 (SAA2); leukotriene B4 receptor (LTB4R); tumor necrosis factor receptor superfamily member 1B (TNFRSF1B); complement component 3a receptor 1 (C3AR1); G protein-coupled receptor 65 (GPR65); baculoviral IAP repeat-containing 1 (BIRC1); CD14 antigen (CD14); serum/glucocorticoid regulated kinase (SGK); CD28 antigen (Tp44) (CD28); apoptosis caspase activation inhibitor (AVEN); TNF receptor-associated factor 3 (TRAF3); aldo-keto reductase family 1, member B10 (AKR1B10); phospholipase A2, group IIA (platelets, synovial fluid) (PLA2G2A); UDP glycosyltransferase 1 family, polypeptide A10 (UGT A10); phospholipid scramblase 1 (PLSCR1); serum amyloid A2 (SAA2); fatty acid binding protein 5 (psoriasis-associated) (FABP5); arachidonate 5-lipoxygenase (ALOX5); phosphoinositide-3-kinase, catalytic, delta polypeptide (PIK3CD); secretoglobin, family 1D, member 2 (SCGB1D2); secretoglobin, family 2A, member 1 (SCGB2A1); transmembrane 4 superfamily member 3 (TM4SF3); mutS homolog 5 (E. coli) (MSH5); secretoglobin, family 2A, member 2 (SCGB2A2); mutS homolog 5 (E. coli) (MSH5); frizzled-related protein (FRZB); hypothetical protein MGC11242 (MGC11242); SRY (sex determining region Y)-box 10 (SOX10); keratin 18 (KRT18); lipase hepatic (LIPC); coagulation factor X (F10); hypothetical protein FLJ20280 (FLJ20280); fast troponin C2, (TNNC2); KDEL endoplasmic reticulum protein retention receptor 3 (KDELR3); KIAA0514; DKFZP5640243 protein; nuclear receptor subfamily 1, group D, member 1 (NR1D1); CD47 antigen (Rh-related antigen, integrin-associated signal transducer) (CD47); Thy1 cell surface antigen (Thy1); selectin P ligand (SELPG); TIMP metallopeptidase inhibitor 1 (TIMP1); chemokine (C-C motif) ligand 21 (CCL21); S100 calcium binding protein A9 (calgranulin B) (S100A9); chemokine (C-C motif) receptor 2 ///chemokine (C-C motif) receptor 2 (CCR2); chemokine (C-C motif) receptor 5 (CCR5); and selectin L (lymphocyte adhesion molecule 1) (SELL).

9. A method of in vitro determination of a patient's sensitivity to developing acne lesions and/or acne associated disorders, the method comprising comparing gene expression or gene expression product levels, or activity of biomarkers selected from the group consisting of matrix metalloproteinase 1 (MMP1); matrix metalloproteinase 3 (MMP3); interleukin 8 (IL8); beta 4defensin (DEFB4); skin-derived (SKALP) protease inhibitor 3 (PI3); chemokine (C-X-C motif) ligand 2 (CXCL2); apolipoprotein B mRNA editing enzyme (APOBEC3A); superoxide dismutase 2, mitochondrial (SOD2); granzyme B (GZMB); S100 calcium binding protein A9 (calgranulin B) (S100A9); chemokine (C-C motif) receptor 1 (CCR1); heparanase (HPSE); serum amyloid A2 (SAA2); leukotriene B4 receptor (LTB4R); tumor necrosis factor receptor superfamily member 1B (TNFRSF1B); complement component 3a receptor 1 (C3AR1); G protein-coupled receptor 65 (GPR65); baculoviral IAP repeat-containing 1 (BIRC1); CD14 antigen (CD14); serum/glucocorticoid regulated kinase (SGK); CD28 antigen (Tp44) (CD28); apoptosis caspase activation inhibitor (AVEN); TNF receptor-associated factor 3 (TRAF3); aldo-keto reductase family 1, member B10 (AKR1B10); phospholipase A2, group IIA (platelets, synovial fluid) (PLA2G2A); UDP glycosyltransferase 1 family, polypeptide A10 (UGT1A10); phospholipid scramblase 1 (PLSCR1); serum amyloid A2 (SAA2); fatty acid binding protein 5 (psoriasis-associated) (FABP5); arachidonate 5-lipoxygenase (ALOX5); phosphoinositide-3-kinase, catalytic, delta polypeptide (PIK3CD); secretoglobin, family 1D, member 2 (SCGB1D2); secretoglobin, family 2A, member 1 (SCGB2A1); transmembrane 4 superfamily member 3 (TM4SF3); mutS homolog 5 (E. coli) (MSH5); secretoglobin, family 2A, member 2 (SCGB2A2); mutS homolog 5 (E. coli) (MSH5); frizzled-related protein (FRZB); hypothetical protein MGC11242 (MGC11242); SRY (sex determining region Y)-box 10 (SOX10); keratin 18 (KRT18); lipase hepatic (LIPC); coagulation factor X (F10); hypothetical protein FLJ20280 (FLJ20280); fast troponin C2, (TNNC2); KDEL endoplasmic reticulum protein retention receptor 3 (KDELR3); KIAA0514; DKFZP5640243 protein; nuclear receptor subfamily 1, group D, member 1 (NR1D1); CD47 antigen (Rh-related antigen, integrin-associated signal transducer) (CD47); Thy1 cell surface antigen (Thy1); selectin P ligand (SELPG); TIMP metallopeptidase inhibitor 1 (TIMP1); chemokine (C-C motif) ligand 21 (CCL21); S100 calcium binding protein A9 (calgranulin B) (S100A9); chemokine (C-C motif) receptor 2 ///chemokine (C-C motif) receptor 2 (CCR2); chemokine (C-C motif) receptor 5 (CCR5); and selectin L (lymphocyte adhesion molecule 1) (SELL) in a patient biological sample with a subject “control” biological sample.

10. A method of preparing a composition for treating acne or an acne associated disorder, the method comprising preparing a composition comprising a modulator of acne lesion biomarkers selected from the group consisting of matrix metalloproteinase 1 (MMP1); matrix metalloproteinase 3 (MMP3); interleukin 8 (IL8); beta 4defensin (DEFB4); skin-derived (SKALP) protease inhibitor 3 (PI3); chemokine (C-X-C motif) ligand 2 (CXCL2); apolipoprotein B mRNA editing enzyme (APOBEC3A); superoxide dismutase 2, mitochondrial (SOD2); granzyme B (GZMB); S100 calcium binding protein A9 (calgranulin B) (S100A9); chemokine (C-C motif) receptor 1 (CCR1); heparanase (HPSE); serum amyloid A2 (SAA2); leukotriene B4 receptor (LTB4R); tumor necrosis factor receptor superfamily member 1B (TNFRSF1B); complement component 3a receptor 1 (C3AR1); G protein-coupled receptor 65 (GPR65); baculoviral IAP repeat-containing 1 (BIRC1); CD14 antigen (CD14); serum/glucocorticoid regulated kinase (SGK); CD28 antigen (Tp44) (CD28); apoptosis caspase activation inhibitor (AVEN); TNF receptor-associated factor 3 (TRAF3); aldo-keto reductase family 1, member B10 (AKR1B10); phospholipase A2, group IIA (platelets, synovial fluid) (PLA2G2A); UDP glycosyltransferase 1 family, polypeptide A10 (UGT A10); phospholipid scramblase 1 (PLSCR1); serum amyloid A2 (SAA2); fatty acid binding protein 5 (psoriasis-associated) (FABP5); arachidonate 5-lipoxygenase (ALOX5); phosphoinositide-3-kinase, catalytic, delta polypeptide (PIK3CD); secretoglobin, family 1D, member 2 (SCGB1D2); secretoglobin, family 2A, member 1 (SCGB2A1); transmembrane 4 superfamily member 3 (TM4SF3); mutS homolog 5 (E. coli) (MSH5); secretoglobin, family 2A, member 2 (SCGB2A2); mutS homolog 5 (E. coli) (MSH5); frizzled-related protein (FRZB); hypothetical protein MGC11242 (MGC11242); SRY (sex determining region Y)-box 10 (SOX10); keratin 18 (KRT18); lipase hepatic (LIPC); coagulation factor X (F10); hypothetical protein FLJ20280 (FLJ20280); fast troponin C2, (TNNC2); KDEL endoplasmic reticulum protein retention receptor 3 (KDELR3); KIAA0514; DKFZP5640243 protein; nuclear receptor subfamily 1, group D, member 1 (NR1D1); CD47 antigen (Rh-related antigen, integrin-associated signal transducer) (CD47); Thy1 cell surface antigen (Thy1); selectin P ligand (SELPG); TIMP metallopeptidase inhibitor 1 (TIMP1); chemokine (C-C motif) ligand 21 (CCL21); S100 calcium binding protein A9 (calgranulin B) (S100A9); chemokine (C-C motif) receptor 2 ///chemokine (C-C motif) receptor 2 (CCR2); chemokine (C-C motif) receptor 5 (CCR5); and selectin L (lymphocyte adhesion molecule 1) (SELL).

11. The method as defined by claim 10, wherein the modulator of acne lesion biomarkers is a modulator of an inflammatory acne lesion biomarker.

12. A method of treating acne or an acne associated disorder, the method comprising administering to an individual subject in need thereof a modulator of acne lesion biomarkers selected from the group consisting of matrix metalloproteinase 1 (MMP1); matrix metalloproteinase 3 (MMP3); interleukin 8 (IL8); beta 4defensin (DEFB4); skin-derived (SKALP) protease inhibitor 3 (PI3); chemokine (C-X-C motif) ligand 2 (CXCL2); apolipoprotein B mRNA editing enzyme (APOBEC3A); superoxide dismutase 2, mitochondrial (SOD2); granzyme B (GZMB); S100 calcium binding protein A9 (calgranulin B) (S100A9); chemokine (C-C motif) receptor 1 (CCR1); heparanase (HPSE); serum amyloid A2 (SAA2); leukotriene B4 receptor (LTB4R); tumor necrosis factor receptor superfamily member 1B (TNFRSF1B); complement component 3a receptor 1 (C3AR1); G protein-coupled receptor 65 (GPR65); baculoviral IAP repeat-containing 1 (BIRC1); CD14 antigen (CD14); serum/glucocorticoid regulated kinase (SGK); CD28 antigen (Tp44) (CD28); apoptosis caspase activation inhibitor (AVEN); TNF receptor-associated factor 3 (TRAF3); aldo-keto reductase family 1, member B10 (AKR1B10); phospholipase A2, group IIA (platelets, synovial fluid) (PLA2G2A); UDP glycosyltransferase 1 family, polypeptide A10 (UGT1A10); phospholipid scramblase 1 (PLSCR1); serum amyloid A2 (SAA2); fatty acid binding protein 5 (psoriasis-associated) (FABP5); arachidonate 5-lipoxygenase (ALOX5); phosphoinositide-3-kinase, catalytic, delta polypeptide (PIK3CD); secretoglobin, family 1D, member 2 (SCGB1D2); secretoglobin, family 2A, member 1 (SCGB2A1); transmembrane 4 superfamily member 3 (TM4SF3); mutS homolog 5 (E. coli) (MSH5); secretoglobin, family 2A, member 2 (SCGB2A2); mutS homolog 5 (E. coli) (MSH5); frizzled-related protein (FRZB); hypothetical protein MGC11242 (MGC11242); SRY (sex determining region Y)-box 10 (SOX10); keratin 18 (KRT18); lipase hepatic (LIPC); coagulation factor X (F10); hypothetical protein FLJ20280 (FLJ20280); fast troponin C2, (TNNC2); KDEL endoplasmic reticulum protein retention receptor 3 (KDELR3); KIAA0514; DKFZP5640243 protein; nuclear receptor subfamily 1, group D, member 1 (NR1D1); CD47 antigen (Rh-related antigen, integrin-associated signal transducer) (CD47); Thy1 cell surface antigen (Thy1); selectin P ligand (SELPG); TIMP metallopeptidase inhibitor 1 (TIMP1); chemokine (C-C motif) ligand 21 (CCL21); S100 calcium binding protein A9 (calgranulin B) (S100A9); chemokine (C-C motif) receptor 2 ///chemokine (C-C motif) receptor 2 (CCR2); chemokine (C-C motif) receptor 5 (CCR5); and selectin L (lymphocyte adhesion molecule 1) (SELL).
